(19) United States
(12) Patent Application Publication
(10) Pub. No.: US 2023/0160900 A1
(43) Pub. Date: May 25, 2023

(54) Method, Apparatus and System for Label-free Testing Whole Blood Specimen Using Fluidic Diffraction Chip

(71) Applicant: NATIONAL TAIWAN UNIVERSITY OF SCIENCE AND TECHNOLOGY, Taipei City (TW)

(72) Inventor: Chien-Kuang CHEN, Taipei City (TW)

(21) Appl. No.: 17/829,216

(22) Filed: May 31, 2022

(30) Foreign Application Priority Data
Nov. 25, 2021 (TW) 110144072

(51) Int. Cl. G01N 33/574; G01N 33/543; G01N 33/569; B01L 3/00; G01N 21/47

(52) U.S. Cl. CPC G01N 33/57492; G01N 33/54306; G01N 33/56911; B01L 3/502761; G01N 21/4788; B01L 2300/0654; B01L 2400/0478; G01N 2201/06113

(57) ABSTRACT

The present invention provides a whole blood sample detection method, device and system using a fluidic diffraction chip, including: injecting a whole blood sample through a diffraction chip; rinsing the diffraction chip; emitting a laser light source through the diffraction chip, wherein the wavelength range of the laser light source is 400 nm to 700 nm, the laser power density range of the diffraction chip is 2 mW/cm² to 2000 mW/cm², and a laser diffraction signal is received on the opposite side of the laser transmitter. The attenuation of the laser diffraction signal calculates the number of a test target. The method, device and system of the present invention can detect the number and status of cells or bacteria without labeling of cells or bacteria.

### CROSSED-REFERENCE TO RELATED APPLICATION

This application claims priority to and the benefit of Taiwan Patent Application No. 110144072, filed on Nov. 25, 2021, in the Taiwan Intellectual Property Office, the entire content of which is incorporated herein by reference.

### FIELD OF THE INVENTION

The present invention relates to a whole blood sample detection method, device and system using a fluidic diffraction chip, and more particularly to a whole blood sample detection method, device and system using a fluidic diffraction chip using laser diffraction technology.

### BACKGROUND OF THE INVENTION

With the development of biochip technology, many tests used to be performed in the laboratory and now can be completed in a small chip, which has the advantages of being lightweight, low cost, and reduced reagent and sample consumption. Although biochip can be used in cell detection, using antibody markers to identify cells, it still requires the step of fluorescent staining and then confirming the number and status of stained cells by fluorescent microscopy. Because white blood cells in the whole blood are often mis-stained and the different fluorescence intensities can easily lead to misjudgment of counts, different dyes and manual methods are needed to eliminate false positives. It would take more than 4 hours to detect a sample, and the detection efficiency does not meet the needs of a large number of patients. Therefore, there is an urgent need for a biological detection method and system that can rapidly and accurately detect the number of cells or bacteria.

### SUMMARY OF THE INVENTION

The present invention provides a method for testing whole blood specimen using diffraction chip comprises: injecting a whole blood sample into a fluidic diffraction chip; rinsing the diffraction chip; emitting a laser light source through the diffraction chip, wherein the wavelength range of the laser light source is 400 nm to 700 nm, and the energy range of the laser light source passing through the diffraction chip is 2 mW/cm² to 2000 mW/cm²; and receiving a laser diffraction signal on the opposite surface of the laser light source, and the cell numbers of a measured target being calculated by the attenuation of the laser diffraction signal, the staining and manual counting does not require during the process.

The system for testing whole blood specimen using diffraction chip comprises: a sample injection component passing a whole blood sample through a diffraction chip; a flushing component for flushing the diffraction chip; a laser transmitter being disposed above a fixing component, which is used for emitting a laser light source through the diffraction chip, wherein the wavelength range of the laser light source is 400 nm to 700 nm, and the laser power density range passing through the diffraction chip is between 2 mW/cm² to 2000 mW/cm²; a laser receiver being disposed below the fixing component and on the opposite side of the laser transmitter, for receiving a laser diffraction signal; a processor receiving the laser diffraction signal and calculating the quantity of a test target according to the attenuation of the laser diffraction signal; and a bearing component being arranged on the fixing component and between the laser transmitter and the laser receiving unit for placing the diffraction chip.

In some of the embodiments, the method further comprises drying the diffraction chip.

In some of the embodiments, the whole blood sample passes through the diffraction chip at a flow rate ranging from 1 ml/hr to 12 ml/hr.

In some embodiment, the diffraction chip comprises: an upper cover, which comprises an input port injecting the whole blood sample into the diffraction chip; and an output port exporting the whole blood sample from the diffraction chip; a chip layer having a diffraction area, wherein the diffraction area comprises a plurality of protrusions, and is grafted with an antibody that specifically binds to the test target; and an adhesive layer, bonding the upper cover and the chip layer, with a thickness of 200 μm to 500 μm, wherein the adhesive layer has a hollow block for the whole blood sample to pass through the diffraction chip, and the hollow block comprises: an injection channel connected to the input port to introduce the whole blood sample into the diffraction area; a diffusion area connecting the injection channel and the vertical line of the diffusion area overlapping the vertical line of the diffusion area; and an outflow channel connected to the diffusion area and connected to the output port to make the whole blood sample flow out of the diffusion area.

In some of the embodiments, the attenuation of the laser diffraction signal is proportional to the quantity of the tested target.

A whole blood sample detection device using a diffraction chip, comprising: a sample injection component including a sample chamber, a syringe pump, an injection channel and an injection joint, wherein the syringe pump controls a whole blood sample in the sample chamber to flow through the injection channel, and the injection joint is set on the injection channel and connected to one of the input ports of a diffraction chip; a flushing component including a flushing solution chamber, a syringe pump and a flushing channel, wherein the syringe pump injects a flushing liquid of the flushing solution chamber into the flushing channel, and the flushing channel communicates with the injection channel; a laser receiver being disposed below the fixing component and on the opposite side of the laser transmitter, for receiving a laser diffraction signal; a processor receiving the laser diffraction signal and calculating the quantity of a test target according to the attenuation of the laser diffraction signal; and a bearing component being arranged on the fixing component and between the laser transmitter and the laser receiving unit for placing the diffraction chip.

In some of the embodiments, the whole blood sample detection device using a diffraction chip further comprises a solution collection component, the solution collection component comprising a collection tank, an export channel and an export joint, wherein two ends of the export channel are connected to the collection tank and the export joint is connected, and the export joint is used for connecting with an output port of the diffraction chip.

In some of the embodiments, the whole blood sample detection device using a diffraction chip further comprises a drying component for drying the diffraction chip.

The whole blood sample detection method using the diffraction chip provided by the present invention can detect the number of the tested target, such as the number of cells or the number of pathogenic bacteria, in the whole blood sample without staining the whole blood sample with fluorescence. Using the laser diffraction technology, through the design of the whole blood sample detection device and system of the present invention, it can be used for various types of cells, especially the test targets such as circulating tumor cells and Yersinia pestis.

### BRIEF DESCRIPTION OF THE DRAWINGS

FIG. 1A is a perspective view of a cylindrical diffraction structure according to an embodiment of the present invention. FIG. 1B is a perspective view of a linear diffraction structure according to an embodiment of the present invention.

FIG. 2 is a flowchart of a whole blood sample detection method according to an embodiment of the present invention.

FIG. 3A is a block diagram of a whole blood sample detection apparatus according to an embodiment of the present invention. FIG. 3B is a block diagram of a whole blood sample detection system according to an embodiment of the present invention.

FIG. 4 is a schematic diagram of a diffraction chip detection system according to an embodiment of the present invention, which comprises a diffraction chip detection device according to an embodiment of the present invention and a diffraction chip used in conjunction therewith.

FIGS. 5A, 5B, 5C and 5D are respectively an exploded view, a combined view, a cross-sectional view, and a top view of a diffraction chip according to an embodiment of the present invention.

FIG. 6 is a distribution diagram of the linear relationship between the number of circulating cells in the tumor and the loss of laser energy and the measurement results of cancer subjects in embodiment 1.

FIG. 7 is a graph showing the relationship between the concentration of Yersinia pestis and the loss of laser energy in embodiment 2.

### DETAILED DESCRIPTION OF THE INVENTION

It should be understood that both the foregoing general description and the following detailed description are exemplary and explanatory and are not intended to limit the scope of the claimed invention. Certain details of one or more embodiments of the invention are set forth in the description below. Other features or advantages of the present invention will be apparent from the following non-exhaustive list of representative embodiments, and also from the scope of the appended claims.

Unless otherwise defined, all technical and scientific terms used herein have the same meaning as commonly understood by one of ordinary skill in the art to which this invention belongs.

As used herein, the articles “a,” “an,” and “any” refer to the grammar of one or more than one (ie, at least one) item. For example, “an element” means one element or more than one element.

As used herein, the terms “around,” “approximately,” or “approximately,” substantially mean that the stated value or range is within 20%, preferably within 10%, and more preferably within 5%. Numerical quantities provided herein are approximations, meaning that they could be inferred if the terms “around,” “approximately,” or “approximately” were not used.

As used herein, the term “label-free” refers to the ability to omit the fluorescent labeling steps common in biological detection, such as but not limited to staining labels such as hematoxylin-eosin staining (H-E Stain) or fluorescent labeling steps such as Cy3 and Cy5.

As used herein, the term “gelatin” refers to a high molecular weight polypeptide derived from collagen, a process that involves structural disruption of native collagen macromolecules, and is a biocompatible material. Type A gelatin is made from acid-pretreated pork skin, and type B gelatin is made from alkali-pretreated bovine bone.

As used herein, the term “thiol self-assembled monolayer” means that a thiol or disulfide derivative can spontaneously form a tightly packed monolayer on a gold surface by self-assembly. The reactivity of gold is relatively passive and the degree of oxidation is small, which is conducive to the adsorption of thiol compounds. Although the high free energy of the gold surface can cause the attachment of hydrocarbons in the air, there is a stronger bond between the thiol functional group and the gold. It is covalently bonded, so it does not affect the film formation.

As used herein, the term “silyl-based self-assembled monolayer” means that 3-aminopropyltriethoxysilane ((3-aminopropyl)triethoxysilane, APTES) undergoes a dealcoholization reaction, —Si—O—C2H5 is hydrolyzed to —Si—OH then adsorbs with the hydroxyl groups on the silicon wafer to form covalent bonds, and aggregates with other APTES molecules adsorbed on the surface to further self-assemble into a spontaneously formed monolayer.

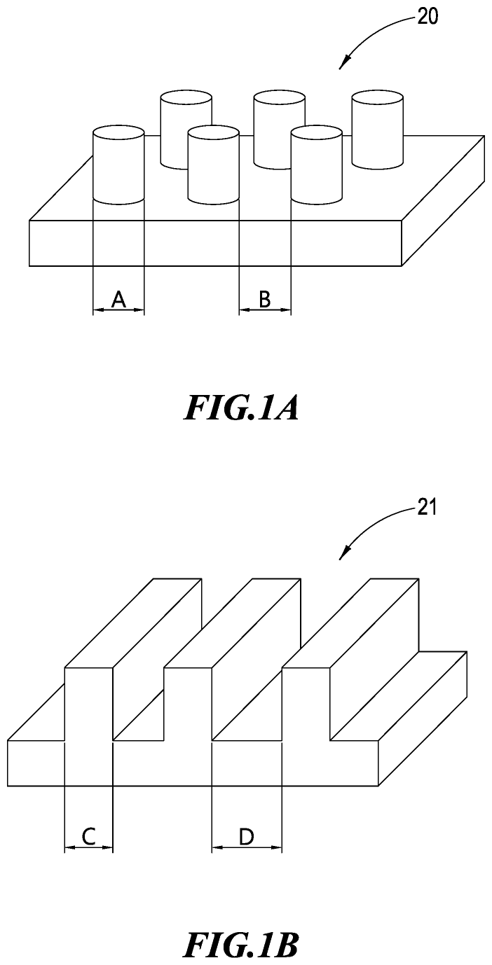

As shown in FIG. 1A, as used herein, the term “column/spacing ratio” refers to the ratio of column width to spacing width. The diffractive structure region of the embodiment of the present invention comprises a plurality of protrusions. When the plurality of protrusions in the diffraction structure are cylindrical protruding parts 20, the distance between the bottoms of the two cylindrical protruding parts 20 is “spacing width” B, and the width of the bottom of the cylindrical protruding part 20 is “column width” A.

As shown in FIG. 1B, as used herein, the term “line/spacing ratio” refers to the ratio of line width to spacing width. The diffractive structure region of the embodiment of the present invention comprises a plurality of protrusions. When the plurality of protrusions in the diffraction structure is linear protruding parts 21, the distance between the bottoms of the two linear protruding parts 21 is the “spacing width” D, and the width of the bottom of the linear protruding parts 21 is “line width” C.

Other technical contents, features and effects of the present invention will be clearly presented in the following detailed description of the preferred embodiments with reference to the drawings.

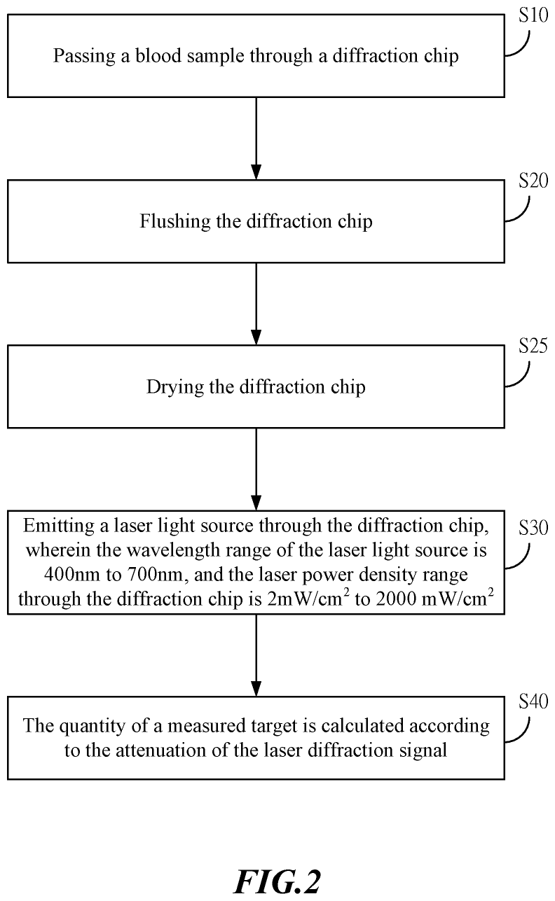

As shown in FIG. 2, an embodiment of the present invention provides a method for detecting a diffraction chip, including passing a whole blood sample through a diffraction chip (step S10); flushing the diffraction chip (step S20); and emitting a laser light source through the diffraction chip, wherein the wavelength range of the laser light source is 400 nm to 700 nm, and the laser power density range through the diffraction chip is 2 mW/cm² to 2000 mW/cm² (step S30); a laser diffraction signal is received on the other side of the laser light source. The quantity of a measured target is calculated according to the attenuation of the laser diffraction signal (step S40).

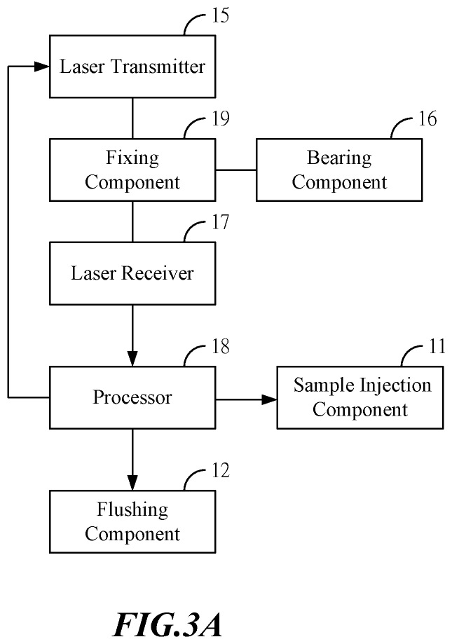

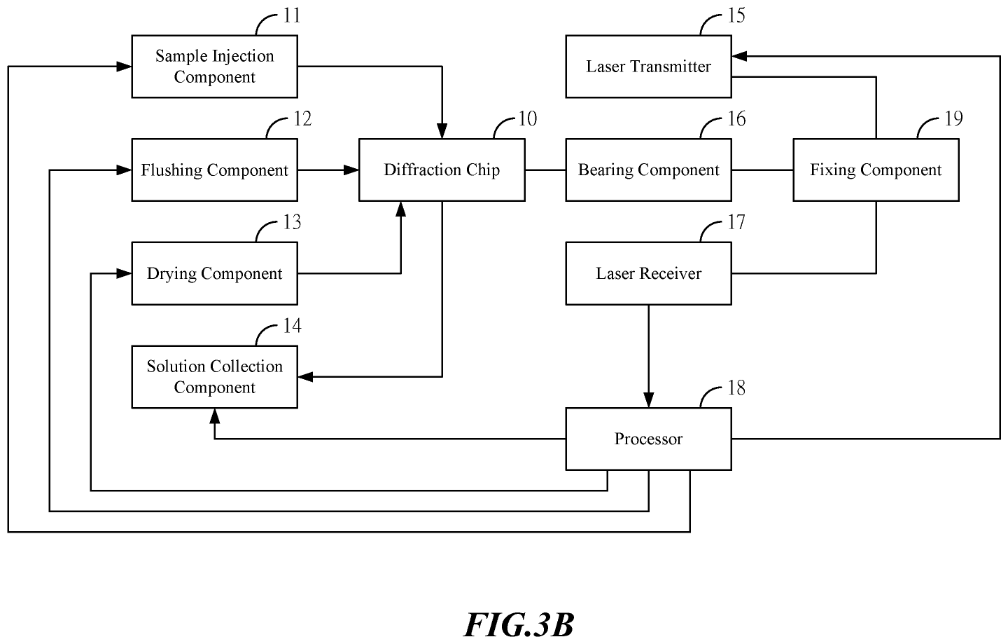

The diffraction chip detection system of the embodiment of the present invention can implement the diffraction chip detection method of the embodiment of the present invention. The diffraction chip detection system 10 according to the embodiment of the present invention comprises a diffraction chip detection device and a diffraction chip used in conjunction therewith. As shown in FIG. 3A, the diffraction chip detection device according to the embodiment of the present invention comprises a sample injection component 11, a flushing component 12, a laser transmitter 15, a laser receiver 17, a bearing component 16, a processor 18 and a fixing component 19. Besides, the diffraction chip detection apparatus according to the embodiment of the present invention may further comprise a drying component 13 and a solution collection component 14. As shown in FIG. 3B, the diffraction chip detection system according to the embodiment of the present invention comprises a sample injection component 11, a flushing component 12, a laser transmitter 15, a laser receiver 17, a bearing component 16, a processor 18, a fixing component 19, a drying component 13, a solution collection component 14 and a diffraction chip 10 that can be used in combination with each other.

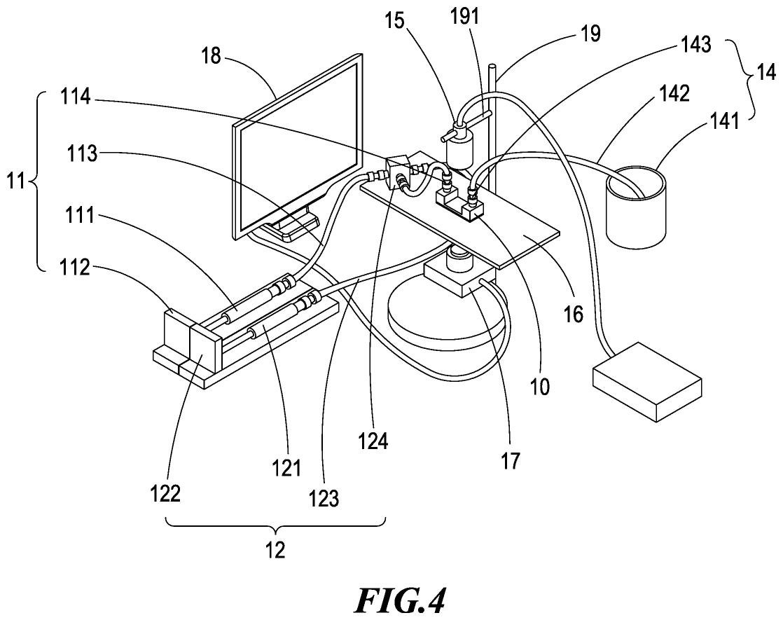

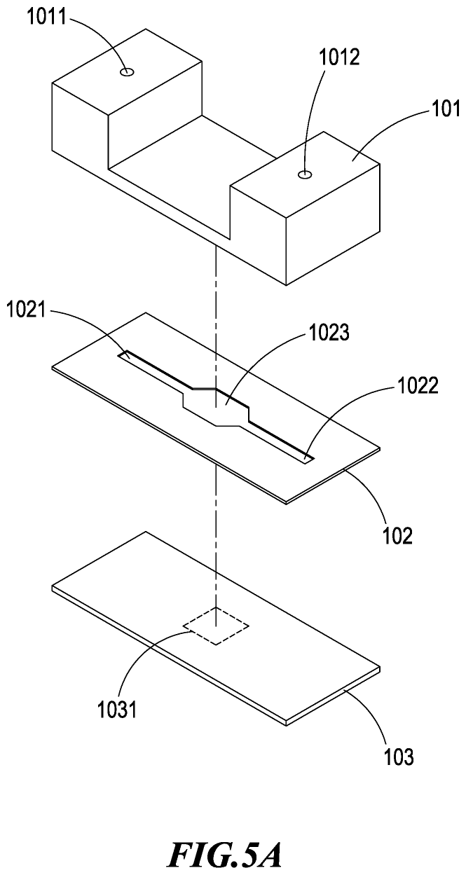

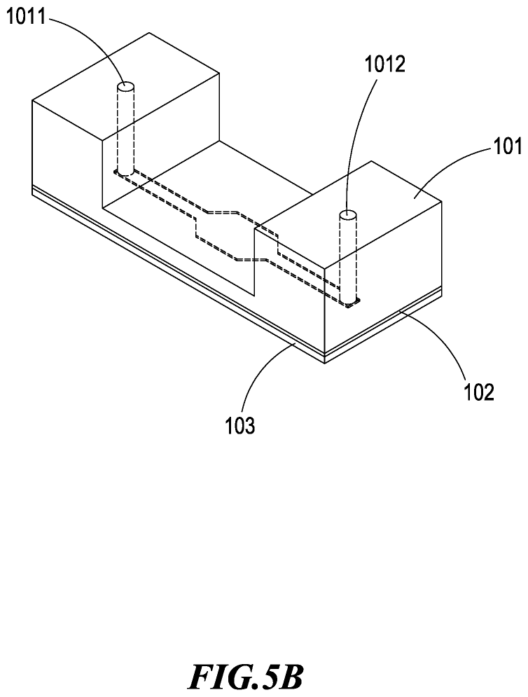

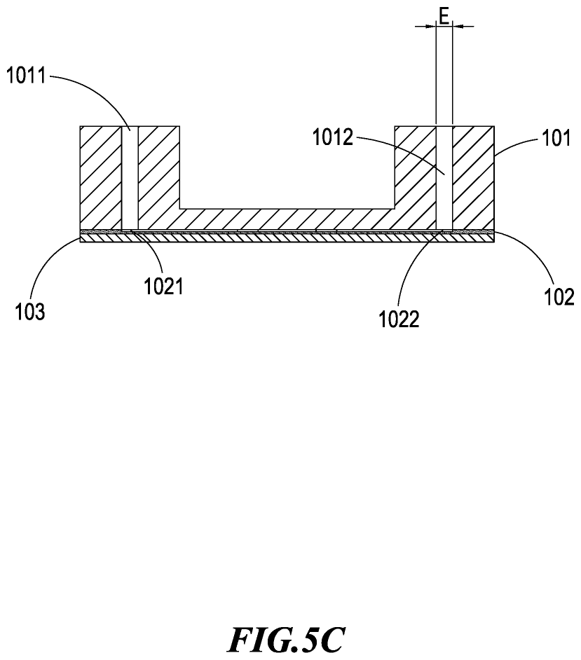

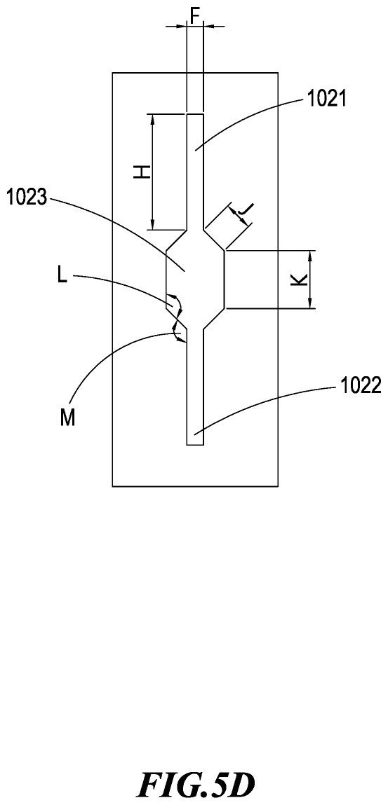

Please refer to FIG. 4, the sample injection component 11 is used to pass a whole blood sample through a diffraction chip 10 (step S10). The sample injection component 11 comprises but is not limited to a manual injection component or an electric injection component with an injection pump, such as a sample chamber 111, a syringe pump 112, an injection channel 113 and an injection joint 114, the sample chamber 111 and the injection joint 114 are located at the start and end of the injection channel 113 respectively, the whole blood sample can be placed in the sample chamber 111, and the syringe pump 112, peristaltic pump or vacuum pump is used to control the whole blood sample injection at the same speed and volume, the whole blood sample is injected into the diffraction chip 10 through the injection channel 113 and through the injection joint 114.

The diffraction chip 10 only requires a small amount of whole blood sample for detection, so sample injection component 11 inject around 1 ml to 3 ml of a whole blood sample for detection, including but not limited to 1 ml, 1.5 ml, 2 ml, 2.5 ml, 3 ml. The rate of infusion of the whole blood sample is related to the capture rate of the test target. The sample injection component 11 inject a whole blood sample at a flow rate ranging from 1 ml/hr to 12 ml/hr, ex, 1 ml/hr, 3 ml/hr, 5 ml/hr, 7 ml/hr, 9 ml/hr, or 11 ml/hr. In certain embodiments, the flow rate can be at any rate in the range of 1 ml/hr to 3 ml/hr, such as 1 ml/hr, 2 ml/hr, or 3 ml/hr, but not limited to integers rate, such as 1.33 ml/hr. In the specific embodiment, the tested target is Yersinia pestis, the rate is preferably 3 ml/hr. In the specific embodiment, the tested target is circulating tumor cells, the rate is preferably 1-2 ml/hr, and the capture rate of circulating tumor cells reaches 70%.

The flushing component 12 is used for flushing the diffraction chip 10 (step S20), and it can also be a manual injection component or an electric injection component. The flushing component 12 comprises a flushing solution chamber 121, a syringe pump 122, a flushing channel 123 and a transfer component 124, wherein the syringe pump 122, a peristaltic pump or a vacuum pump injects a flushing solution from the flushing solution chamber 121 into the flushing channel 123, and the flushing channel 123 communicates with the injection channel 113, preferably the flushing channel 123 can be introduced into the injection channel 113 through the transfer component 124, such as a three-way joint or a three-way valve. A section of the injection channel 113 is formed between two joints of the three-way joint, and one joint is connected to the flushing channel 123. After the test target in the whole blood sample is captured, it requires to be washed with Phosphate buffered saline (PBS) to prevent other substances on the diffraction chip 10 from affecting the test results. The washing speed is preferably 10 ml/min, the flush volume is preferably 10 ml. In addition, it is better to set the transfer component 124 between the injection channel 113, the sample chamber 111 and the flushing channel 123, so that when different whole blood samples are detected, all channels can be flushed to avoid contamination between different samples.

After rinsing, the diffraction chip 10 needs to be dried to obtain more accurate detection results. Therefore, the whole blood sample detection device and system according to the embodiments of the present invention may further comprise a drying component 13 to improve the detection speed. The drying component 13 may be an air blowing device or a fan device. For drying or blowing the diffraction chip 10 (step S25). The solution collection component 14 comprises but is not limited to a separable collection tank 141, an export channel 142 and an export joint 143. Both ends of the export channel 142 communicate with the collection tank 141 and the export joint 143, wherein the collection tank 141 can be a vacuum tank, and the solution collection component 14 can be used to collect the whole blood sample and phosphate-buffered saline injected into the flushing component 12 after passing through the diffraction chip 10.

The laser transmitter 15 is disposed on the first position fixing component 191 above the fixing component 19 for emitting a laser light source through the diffraction chip 10 (step S30). The laser transmitter 15 comprises, but is not limited to a laser with a laser light source with a wavelength range of around 400 nm to 700 nm, and a laser power density range of around 2 mW/cm² to 2000 mW/cm² through the diffraction chip 10. The laser 15 can adjust the appropriate energy according to different situations. In certain embodiments, the wavelength of the laser is 425 nm, 450 nm, 475 nm, 500 nm, 525 nm, 550 nm, 575 nm, 600 nm, 625 nm, 650 nm, 675 nm, or 700 nm. In some specific embodiments, the laser light source is a green light with a wavelength of 523 nm. Laser power density through diffraction chip 10 comprises but is not limited to 5 mW/cm², 15 mW/cm², 25 mW/cm², 35 mW/cm², 45 mW/cm², 55 mW/cm², 65 mW/cm², 75 mW/cm², 85 mW/cm², 95 mW/cm², 105 mW/cm², 205 mW/cm², 305 mW/cm², 405 mW/cm², 505 mW/cm², 805 mW/cm², 1105 mW/cm², 1405 mW/cm² or 1705 mW/cm², wherein a filter can be added to the laser transmitter 15 to adjust the appropriate laser energy.

The laser receiver 17 is arranged on a third position fixing member of the fixing component 19 and is located on the opposite surface of the laser transmitter 15, so that the laser receiver 17 and the center of the laser transmitter 15 are located on the same axis to directly receive the output from the laser transmitter 15 and pass through the diffraction chip 10, then attenuates a laser diffraction signal (step S40). The laser receiver 17 comprises but is not limited to an array beam laser analyzer or a CCD (Charge-coupled Device) laser camera, and the distance from the laser receiver 17 is around 5 cm to 30 cm, including but not limited to 5 cm, 10 cm, 15 cm, 20 cm, 25 cm or 30 cm. The array beam laser analyzer measures the divergence angle of semiconductor lasers by the indirect measurement method, which can accurately obtain the spatial distribution of the spot intensity, and can also draw the long-axis and short-axis distribution curves to calculate the far-field divergence angle of the laser.

The fixing component 19 comprises a first position fixing component 191, a second position fixing member (figure not shown), and a third position fixing member (figure not shown), wherein the first position fixing component 191, the second position fixing member and the third position fixing member It is set in order from top to bottom. The bearing component 16 comprises but is not limited to a carrier, which is disposed on the second position fixing member of the fixing component 19 and located between the laser transmitter 15 and the laser receiver 17 for placing the diffraction chip 10 so that the laser light passes through the diffraction chip 10 and can be detected by the laser receiver 17. The distance between the first position fixing component 191 and the second position fixing member is around 3 cm to 10 cm, and the distance between the second position fixing member and the third position fixing member is around 3 cm to 10 cm. In addition, the bearing component 16 may further comprise a conveying member and a clamping member, the conveying member is connected to the carrier and transports the diffraction chip 10 from a wafer inlet of the diffraction chip detection device to the carrier, and the clamping member is on the carrier. The top of the table is used to fix the diffraction chip 10.

The processor 18, such as a microprocessor, a computer device, or a tablet device, is used to receive the laser diffraction signal and calculate the quantity of a test target according to the attenuation of the laser diffraction signal (step S40). The diffraction area on the diffraction chip 10 will generate a diffraction grating. The object under test will change its surface characteristics after being captured by the diffraction chip 10, the greater the number of objects under test, the more laser energy is absorbed, so the laser diffraction signal is attenuated. Taking the laser energy received before the whole blood sample as the reference value, the attenuation of the laser energy after passing the whole blood sample can be known, and the number of tested targets and the corresponding laser attenuation can be drawn as a trend line. Then, the attenuation of the laser diffraction signal of the trend line is proportional to the number of the tested target, and the measured laser attenuation can be used to estimate the amount of the tested target, which can be used for cell growth monitoring or the positive or negative judgement of cancer test. Laser energy loss=(the energy measured by the laser through the diffraction chip−the energy measured by the laser through the diffraction chip that has grabbed the target)/the energy measured by the laser through the diffraction chip. The term “energy” in the above formula comprises the use of laser power density, laser energy density, or a value measured by a laser receiver to represent energy.

In addition, in an embodiment of a commercial diffraction chip detection system, the processor 18 can be used to further automate the process design of the sample injection component 11, flushing component 12, drying component 13, solution collection component 14 or laser transmitter 15. The above units can be designed to be installed in housing for automated detection. On the user interface, the detection process, the flow rate or flow rate or time of the sample injection component 11 or flushing component 12, the drying time of the drying component 13, the automatic discharge timing or reminder timing of the solution collection component 14, and the energy of the laser transmitter 15 can be set respectively.

As shown in FIGS. SA to 5D, the length of the diffraction chip 10 is around 3 cm to 15 cm, such as 3 cm, 5 cm, 7 cm, 9 cm, 11 cm, 13 cm, or 15 cm. The width is 1 cm to 5 cm, such as 1 cm, 3 cm, or 5 cm. The diffraction chip 10 comprises an upper cover 101, a chip layer 103 and an adhesive layer 102. The upper cover 101 comprises an input port 1011 and an output port 1012, and the input port 1011 and the output port 1012 penetrate from the top of the upper cover 101 to the bottom of the upper cover 101. The adhesive layer 102 has a hollow block, wherein the hollow block comprises an injection channel 1021, a diffusion area 1023 and an outflow channel 1022.

The whole blood sample is injected into the diffraction chip 10 from the input port 1011, wherein the input port 1011 can be connected with the injection joint 114 of the sample injection component 11. The bottom port diameter E of port 1011 is around 0.08 cm to 0.4 cm. The output port 1012 exports the whole blood sample or flushing solution out of the diffraction chip 10 and is connected to the export joint 143 of the solution collection component 14. The material of the upper cover 101 comprises but is not limited to acrylic (poly (methyl methacrylate), PMMA). The thinner the area between the input port 1011 and the output port 1012, the better to reduce the attenuation caused by the passing of the laser.

The thickness of the chip layer 103 ranges from around 300 μm to 500 μm, such as 300 μm, 350 μm, 400 μm, 450 μm, and 500 μm, and has a diffraction area 1031. The diffraction area 1031 comprises a plurality of protrusions, the protrusions can be columnar or linear to form a diffractive structure, wherein column/space ratio or line/space ratio may be around 1:1 to 1:1.5, column width and line width could be around 500 nm, 1 μm. The chip layer 103 can be made of polydimethylsiloxane (PDMS), poly (methyl methacrylate) (PMMA) or polyethylene terephthalate (PET), on which the diffractive columnar or linear protrusions are transferred by a lithography process. Diffraction area 1031 further contains self-assembled monolayer, protein G and antibody. In addition, a release layer may be further comprised between the self-assembled monolayer and the protein G, or a primary antibody adhesion layer may be further comprised after grafting an antibody that specifically binds to the test target.

The self-assembled monolayer comprises, but is not limited to, a thiol self-assembled monolayer and a silane-based self-assembled monolayer, formed on the upper layer of the diffraction structure. On the self-assembled molecular layer, 1-ethyl-3-(3-dimethylaminopropyl) carbodiimide/N-hydroxysuccinimide (EDC/NHS) reaction can be used to engage protein G. Protein G is a cell wall protein isolated from G-type Streptococcus can be used as a bridge to connect with antibodies. Selecting a specific antibody can specifically bind to the test target, thereby complementing the test target. For example, epithelial cell adhesion molecule antibody (Anti-EpCAM) can specifically bind to circulating tumor cells, so it can be grafted on protein G Anti-EpCAM to detect circulating tumor cells. In addition, after the antibody surface is modified, an anti-adhesion layer can be further formed thereon, for example, using bovine serum albumin (Bovine Serum Albumin, BSA) to clean the surface to achieve the anti-adhesion effect of non-specific proteins.

The traditional fluorescently stained test target cannot be used for other research or detection after the detection. However, the diffraction chip 10 of the embodiment of the present invention can be used for rapid screening. On the other hand, the captured test target can be released for subsequent research or other detection, for example, physicians can observe the morphology of cells or bacteria as a basis for medication and treatment so as to provide a wider range of uses. The implementation method is also very easy and only requires further including a release layer between the self-assembled monolayer and protein G, and the release layer material comprises, but is not limited to gelatin or alginic acid. The self-assembled monolayer can be formed on the self-assembled monolayer by the combination of covalent bonds through the EDC/NHS reaction. Therefore, the bond of the release layer can be broken by using alginate lyase, and the test target can be taken out.

Adhesive Layer 102 is used to bond the upper cover 101 and the chip layer 103. Double-sided tape with a thickness of around 200 μm to 500 μm can be used, and a hollow block can be cut on the double-sided tape to set a flow path for the whole blood sample scope. The hollow block of the adhesive layer 102 comprises the injection channel 1021, the diffusion area 1023 and the outflow channel 1022. The injection channel 1021 is connected to the input port 1011, the width F of the injection channel 1021 is around 0.04 cm to 0.2 cm, and the length H is around 0.32 cm to 1.6 cm. The diffusion area 1023 introduces the whole blood sample into the diffusion area 1031, and through two sides of the hexagonal design, the side length range J is around 0.12 cm to 0.6 cm, the lead angle range (L/M) is 91 degrees to 150 degrees, and the whole blood sample is completely guided to flow through the square diffraction area 1031, the side length range K is around 0.2 cm to 1.0 cm, and the whole blood sample flowing through the diffraction area 1031 is received back into the outflow channel 1022 by the other two sides of the hexagonal design. The input and output parts of the hollow block are symmetrically designed. The outflow channel 1022 is connected to the output port 1012 and allows the whole blood sample to flow out of the diffraction area 1031. Such a design can achieve the whole process of sealing to avoid the outflow of whole blood samples. Meanwhile, the diffraction chip 10 is disposable, which is cheap and can be mass-produced.

### Embodiment 1. Evaluation of Endometrial Cancer

The diffraction chip is a PDMS substrate transferred with a diffraction pattern with a column/spacing ratio of 1:1.5 (the diameter of the bottom of the column is 500 nm). After gold plating, immerse in prepared 30 mM Thioglycolic acid (TA) aqueous solution (40 μL+20 mL H2O) for one hour to form a thiol self-assembled monolayer on the PDMS substrate. Then the diffraction chip is rinsed with deionized water 3 times and dried in a nitrogen atmosphere to form PDMS-TA. 3 mg A-type gelatin (Sigma-Aldrich, USA), 35 μL EDC (Alfa Aesar, USA) and 25 mg NHS (Acros Organics, Belgium) were added to 1 mL deionized aqueous solution and reacted with PDMS-TA at 37° C. After one hour, the gelatin grafting was completed by rinsing with deionized water 3 times to become PDMS-TA-Gel.

Next, 75 μL of EDC, 50 mg of NHS and 100 μg of protein G (Bio vision, USA) were added to 1 mL of PBS solution with pH=7.2˜7.4, and reacted with PDMS-TA-Gel at 37° C. for one hour. Rinse 3 times with PBS, and then add 0.02 μg/ml Anti-EpCAM to react for one hour to successfully modify the antibody on the diffraction chip. Rinse 3 times with PBS to remove excess Anti-EpCAM. Then, immersed in bovine serum albumin (Sigma-Aldrich, USA) solution for 30 minutes and rinsed 3 times with PBS to form an anti-adhesion layer against non-specific proteins. Finally, use double-sided adhesive tape with a thickness of around 200 μm to attach an acrylic plate cover with a thickness of around 0.1 cm in the central area (excluding the output port and input port) to form a diffraction chip with a length of around 3.5 cm and a width of around 1.5 cm. The diameter of the port and output port E is around 0.2 cm, the thickness of the chip layer is around 400 μm, and the diffraction area is around 0.5 cm long and 0.5 cm wide. In the hollow area of the double-sided tape, the H range is around 0.8 cm, the F range is around 0.1 cm, the K range is around 0.5 cm, the J range is around 0.3 cm, and the L and M ranges are around 135 degrees.

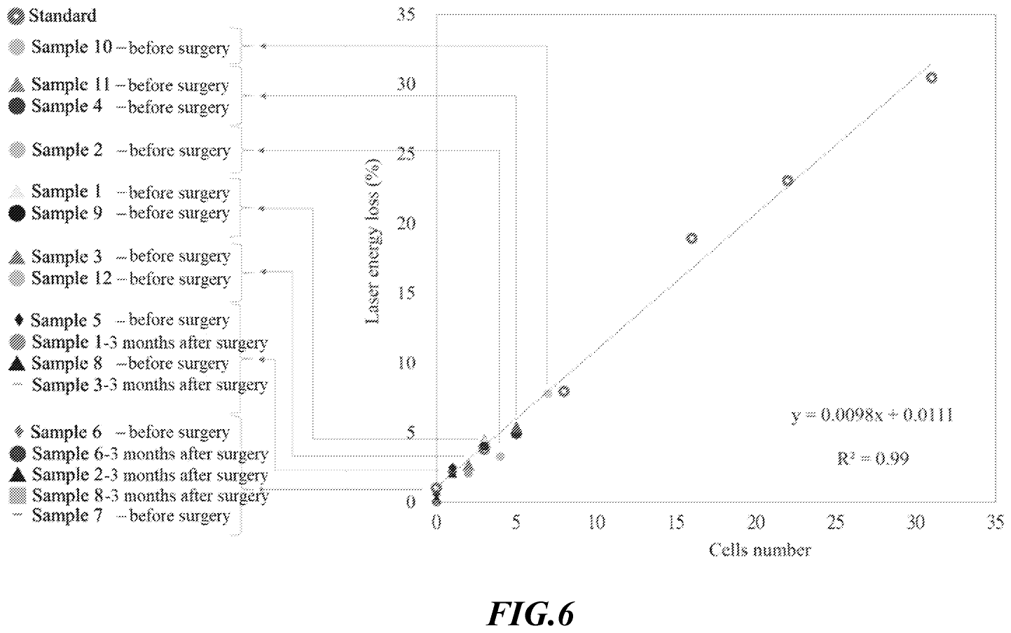

As shown in FIG. 6, put different numbers of cancer cells through the diffraction chip, and detect the corresponding laser energy loss (%), and draw the above results as a trend line. The equation between laser energy loss and captured cells is: y=0.0098x+0.0111, where y is the laser energy loss (%), x is the number of captured cells, and the linear correlation coefficient R2=0.99, showing a high linear relationship. Next, in this embodiment, fresh whole blood was collected from 12 clinical endometrial cancer patients, and some patients had sampling before and after 3 months of surgery. Each whole blood sample was taken 3 mL into a syringe and injected with a pump (CHEMXY Fusion 100) to pass the whole blood sample through the diffraction chip at a flow rate of 3 ml/hr. A laser with a wavelength of 532 nm and a power density of 50 mW/cm² is used, and the distance between the laser transmitter and the stage is around 10 cm, and the distance between the stage and the laser receiver is around 10 cm. When the laser vertically passes through the microstructure on the diffraction chip, the laser diffraction will occur. The array laser beam analyzer BeamMic (UNICE E-O Service, Taiwan) on the opposite side of the laser transmitter is used to measure the energy loss of laser diffraction (The initial measured energy was around 75 Mcnt), and the number of captured circulating tumor cells was estimated based on the laser energy loss. Among the 11 tested samples, 10 of them captured circulating tumor cells, and the number was between 1 and 7. However, the traditional tumor index CA125 detection only detected 3 higher than the reference value of 35 unit/ml. Obviously, the detection method, device and system of the first embodiment of the present invention have a higher detection accuracy than the tumor index CA125 detection method, and compared with the traditional fluorescence detection method, it can show fast screening results without going through a fluorescent labeling procedure and waive the estimate the number of cells.

### Embodiment 2. The Capture and Release of Yersinia pestis

The diffraction chip is nano-imprinted on PET film with a patterned silicon wafer mold with a line/space ratio of 1:1.5 (the width of the bottom of the line is 500 nm), and the surface plasma activation is performed with an oxygen plasma machine power of 100 W for 5 minutes, and then soaked in a 0.05% (v/v) APTES aqueous solution (10 μL+20 mL H2O) for 10 minutes to form a silane-based self-assembled monolayer on the PET substrate, then washed with a large amount of pure water and dried in a nitrogen atmosphere. Prepare 0.5 mg/ml alginic acid (Acros Organics, Belgium) solution, add 35 μg EDC and 25 μg NHS to 200 μL alginic acid aqueous solution, react with PET substrate at 37° C. for 30 minutes, and wash 3 times with PBS.

Next, take 35 μL of EDC, 25 mg NHS and 60 μg protein G into 1000 mL of PBS solution with pH=7.2˜7.4, react with the substrate at 37° C. for one hour, rinse with PBS three times, and then add 0.02 μg/ml of Yersinia pestis antibody (Anti-Yp F1MoAB) was reacted for one hour to successfully modify the antibody on the diffraction chip, and the excess Anti-Yp F1MoAB was removed by rinsing with PBS three times. Then, soak in bovine serum albumin solution (5% in PBS) for one hour, and rinse with PBS three times to form an anti-adhesion layer against non-specific proteins. Finally, the cover of the acrylic plate is attached with double-sided tape (PMMA) to form a diffraction chip. Please refer to the embodiment 1 for the dimension parameters and device settings of other chips.

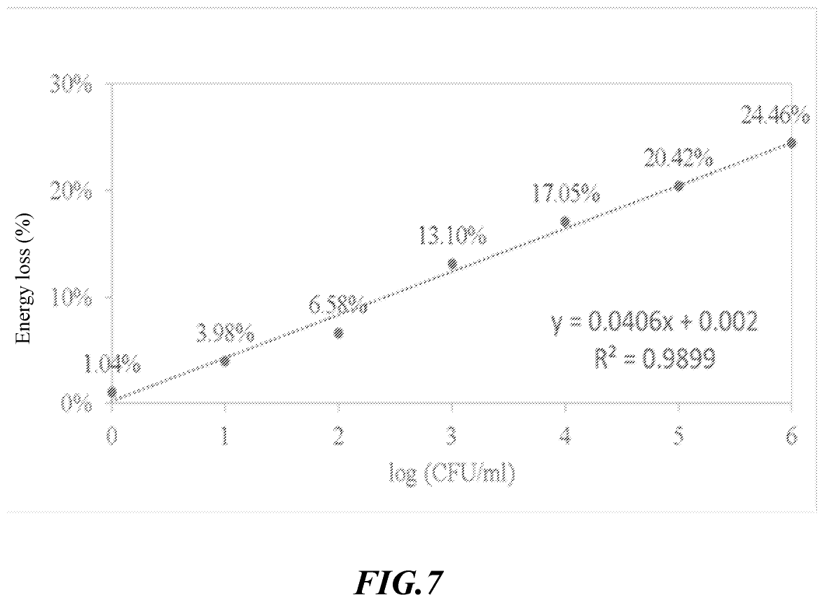

As shown in FIG. 7, different concentrations of Yersinia pestis solutions of 102˜106 CFU/ml were used in this experiment. 1 mL of Yersinia pestis solutions were loaded into syringes, and the whole blood samples were injected with a pump (CHEMXY Fusion 100) at a flow rate of 1 ml/hr to pass through the diffraction chip, then rinsed with PBS and dried with nitrogen. A laser with a wavelength of 532 nm and a power density of 50 mW/cm² is used, when the laser passes vertically through the microstructure on the diffraction chip, the laser diffraction will occur. Use the array laser beam analyzer BeamMic (Honghui Optoelectronics, Taiwan) to measure the laser energy loss on the opposite side of the laser transmitter, and use the laser energy loss to calculate the calibration line. At the same time, the results were confirmed by a fluorescence microscope. The equation between the measured laser energy loss and the bacteria captured is y=0.0406x+0.002, where y is the laser energy loss (%), x is log(CFU/ml), and the linear correlation coefficient R²=0.9899, which shows a highly linear relationship. Meanwhile, after the PET substrate is reacted with alginate lyase, the alginic acid macromolecule is detached with the bacteria to complete the release effect, and the activity of the detached bacteria is not affected, and other follow-up research and testing can be carried out.

The above-detailed description is a specific description of a feasible embodiment of the present invention, but this embodiment is not intended to limit the patent scope of the present invention. Any equivalent implementation or modification that does not depart from the technical spirit of the present invention shall be comprised within the scope of the patent in this case.

What is claimed is:

1. A method for testing whole blood specimen using fluidic diffraction chip comprising:
injecting a whole blood sample into a diffraction chip;
rinsing the diffraction chip;
emitting a laser light source through the diffraction chip, wherein the wavelength range of the laser light source is 400 nm to 700 nm, and the energy range of the laser light source passing through the diffraction chip is 2 mW/cm² to 2000 mW/cm²; and receiving a laser diffraction signal on the opposite surface of the laser light source, and the quantity of a measured target being calculated by the attenuation of the laser diffraction signal.

2. The method for testing whole blood specimen using diffraction chip of claim 1 further comprising drying the diffraction chip.

3. The method for testing whole blood specimen using diffraction chip of claim 1, wherein the whole blood sample passes through the diffraction chip at a flow rate ranging from 1 ml/hr to 12 ml/hr.

4. The method for testing whole blood specimen using diffraction chip of claim 3, wherein the whole blood sample passes through the diffraction chip at a flow rate ranging from 1 ml/hr to 3 ml/hr.

5. The method for testing whole blood specimen using diffraction chip of claim 3, wherein the wavelength range of the laser light source is 500 nm to 575 nm, and the laser power density range passing through the diffraction chip is between 10 mW/cm² to 100 mW/cm²

6. The method for testing whole blood specimen using diffraction chip of claim 3, wherein the diffraction chip comprises:
an upper cover, which comprises an input port injecting the whole blood sample into the diffraction chip; and an output port exporting the whole blood sample from the diffraction chip;
a chip layer having a diffraction area, wherein the diffraction area comprises a plurality of protrusions, and is grafted with an antibody that specifically binds to the test target; and
an adhesive layer, bonding the upper cover and the chip layer, with a thickness of 200 μm to 500 μm, wherein the adhesive layer has a hollow block for the whole blood sample to pass through the diffraction chip, and the hollow block comprises:
an injection channel connected to the input port to introduce the whole blood sample into the diffraction area;
a diffusion area connecting the injection channel and the vertical line of the diffusion area overlapping the vertical line of the diffusion area; and
an outflow channel connected to the diffusion area and connected to the output port to make the whole blood sample flow out of the diffusion area.

7. The method for testing whole blood specimen using diffraction chip of claim 1, wherein the attenuation of the laser diffraction signal is proportional to the quantity of the tested target.

8. A system for testing whole blood specimen using diffraction chip comprising:
a sample injection component for passing a whole blood sample through a diffraction chip;
a flushing component for flushing the diffraction chip;
a laser transmitter being disposed above a fixing component, is used for emitting a laser light source through the diffraction chip, wherein the wavelength range of the laser light source is 400 nm to 700 nm, and the laser power density range passing through the diffraction chip is between 2 mW/cm² to 2000 mW/cm²;
a laser receiver being disposed below the fixing component and on the opposite side of the laser transmitter, for receiving a laser diffraction signal;
a processor receiving the laser diffraction signal and calculating the quantity of a test target according to the attenuation of the laser diffraction signal; and
a bearing component being arranged on the fixing component and between the laser transmitter and the laser receiving unit for placing the diffraction chip.

9. The system for testing whole blood specimen using diffraction chip of claim 8 further comprising a drying component for drying the diffraction chip.

10. The system for testing whole blood specimen using diffraction chip of claim 8, wherein the sample injection component injects the whole blood sample at a flow rate ranging from 1 ml/hr to 12 ml/hr.

11. The system for testing whole blood specimen using diffraction chip of claim 10, wherein the sample injection component injects the whole blood sample at a flow rate ranging from 1 ml/hr to 7 ml/hr.

12. The system for testing whole blood specimen using diffraction chip of claim 10, wherein the diffraction chip comprises:
an upper cover, which comprises an input port injecting the whole blood sample into the diffraction chip; and an output port exporting the whole blood sample from the diffraction chip;
a chip layer having a diffraction area, wherein the diffraction area comprises a plurality of protrusions, and is grafted with an antibody that specifically binds to the test target; and
an adhesive layer, bonding the upper cover and the chip layer, with a thickness of 200 μm to 500 μm, wherein the adhesive layer has a hollow block for the whole blood sample to pass through the diffraction chip, and the hollow block comprises:
an injection channel connected to the input port to introduce the whole blood sample into the diffraction area;
a diffusion area connecting the injection channel and the vertical line of the diffusion area overlapping the vertical line of the diffusion area; and
an outflow channel connected to the diffusion area and connected to the output port to make the whole blood sample flow out of the diffusion area.

13. The system for testing whole blood specimen using diffraction chip of claim 8, wherein the attenuation of the laser diffraction signal is proportional to the quantity of the tested target.

14. The system for testing whole blood specimen using diffraction chip of claim 8, wherein the sample injection component comprises a sample chamber, a syringe pump, an injection channel and an injection joint, wherein the syringe pump of the sample injection component controls the whole blood sample in the sample chamber to flow through the injection channel, and the injection joint is set on the injection channel and connected to one of the input ports of the diffraction chip.

15. The system for testing whole blood specimen using diffraction chip of claim 14, wherein the flushing component comprises a flushing solution chamber, a syringe pump and a flushing channel, wherein the syringe pump of the flushing component injects a flushing liquid of the flushing solution chamber into the flushing channel, and the flushing channel communicates with the injection channel.

16. The system for testing whole blood specimen using diffraction chip of claim 15 further comprising a solution collection component, the solution collection component comprising a collection tank, an export channel and an export joint, wherein two ends of the export channel are connected to the collection tank and the export joint is connected, and the export joint is used for connecting with an output port of the diffraction chip.

17. The system for testing whole blood specimen using diffraction chip of claim 16 further comprising a drying component for drying the diffraction chip.

18. A whole blood sample detection device using a diffraction chip, comprising:
a sample injection component including a sample chamber, a syringe pump, an injection channel and an injection joint, wherein the syringe pump controls a whole blood sample in the sample chamber to flow through the injection channel, and the injection joint is set on the injection channel and connected to one of the input ports of a diffraction chip;
a flushing component including a flushing solution chamber, a syringe pump and a flushing channel, wherein the syringe pump injects a flushing liquid of the flushing solution chamber into the flushing channel, and the flushing channel communicates with the injection channel;
a laser receiver being disposed below the fixing component and on the opposite side of the laser transmitter, for receiving a laser diffraction signal;
a processor receiving the laser diffraction signal and calculating the quantity of a test target according to the attenuation of the laser diffraction signal; and
a bearing component being arranged on the fixing component and between the laser transmitter and the laser receiving unit for placing the diffraction chip.

19. The whole blood sample detection device using a diffraction chip of claim 18 further comprising a solution collection component, the solution collection component comprising a collection tank, an export channel and an export joint, wherein two ends of the export channel are connected to the collection tank and the export joint is connected, and the export joint is used for connecting with an output port of the diffraction chip.

20. The whole blood sample detection device using a diffraction chip of claim 19 further comprising a drying component for drying the diffraction chip.
